(19) United States
(12) Patent Application Publication
(10) Pub. No.: US 2024/0383809 A1
(43) Pub. Date: Nov. 21, 2024

(54) COMPOSITE ARCHITECTURAL ULTRA-HIGH PERFORMANCE CONCRETE MIXTURES AND PANELS WITH ENHANCED PHOTOCATALYTIC ACTIVITY

(71) Applicant: TAKTL LLC, Turtle Creek, PA (US)

(72) Inventor: George Quercia, Cheswick, PA (US)

(73) Assignee: TAKTL LLC, Turtle Creek, PA (US)

(21) Appl. No.: 18/620,345

(22) Filed: Mar. 28, 2024

Related U.S. Application Data
(60) Provisional application No. 63/455,683, filed on Mar. 30, 2023.

(51) Int. Cl. C04B 22/06; A61L 2/23; B01J 21/06; B01J 35/39; B01J 35/61; B28B 23/00; C04B 14/04; C04B 14/08; C04B 14/10; C04B 14/22; C04B 28/02; C04B 111/00; C04B 111/20; C04B 111/80

(52) U.S. Cl. CPC C04B 22/06; A61L 2/23; B01J 21/063; B01J 35/39; B01J 35/613; B01J 35/615; B28B 23/0081; C04B 14/045; C04B 14/08; C04B 14/106; C04B 14/22; C04B 28/02; C04B 2111/00827; C04B 2111/2061; C04B 2111/802

(57) ABSTRACT

A composite ultra-high-performance concrete mixture includes cement in an amount between 500 and 680 kg/m³; a photocatalyst in amount between 10 and 30 kg/m³; a recycled glass white nano-silica in amount 40 and 60 kg/m³; calcinated kaolin in amount 70 to 120 kg/m³; a filler in amount between 200 and 300 kg/m³; a fine sand in amount between 600 to 970 kg/m³; and a coarse sand in amount between 400 to 600 kg/m³.

### CROSS-REFERENCE TO RELATED APPLICATION

Benefit is claimed of U.S. Patent Application No. 63/455,683, filed Mar. 30, 2023, and entitled Composite Architectural Ultra-High Performance Concrete mixtures and panels with Enhanced Photocatalytic activity, the disclosure of which is incorporated by reference herein in its entirety as if set forth at length.

### BACKGROUND OF THE INVENTION

The invention relates to composite ultra-high performance concrete compositions containing a photocatalyst, a recycled glass white nano-silica and lower cement content. The disclosure also relates to a method for producing thin-walled composites UHPPC facade panels and elements for building envelopes with enhanced photocatalytic activity by the use of clear glass and marble face decorative aggregates.

Photocatalysts are known to enhance activity for desired reactions, including with respect to building materials. However, in traditional concrete applications, photocatalytic activity is quickly deactivated. Therefore, the need remains for a concrete composition which includes a photocatalyst wherein the photocatalyst has extended catalytic activity.

### SUMMARY OF THE INVENTION

The present disclosure provides a UHPC concrete including a photocatalyst wherein the photocatalyst retains activity for prolonged periods of time.

It has been found that a UHPC matrix such as is disclosed in U.S. Pat. No. 11,542,198 assigned to the present applicant, reduces the amount of binder carbonation that the photocatalyst is exposed to. Carbonation can be responsible for photocatalyst deactivation in conventional cementitious materials (normal concrete and mortars). U.S. Pat. No. 11,542,198 is incorporated herein by reference as though set forth herein in its entirety.

Further, the use of amorphous white nano-silica decreases free portlandite and induces micro- and meso-porosity on the surface that enhances the photocatalytic reaction.

The use of calcinated metakaolin produces C-A-S-H type gels that retain more water than standard C-S-H gel, enhancing the photocatalytic reaction at the surface.

The use of glass clear aggregates enhances the light distribution through a panel and allows photocatalytic particles under the panel surface to become active (FIG. 2) and to avoid deactivation due to the formation of nitrates salts on the photocatalytic active center.

The use of marble provides Ca+ and mineralization of nitrates (NO₃⁻) or carbonate species on the surface which are more soluble in water, avoiding deactivation of the photocatalyst and generation of undesired NO₂ by-product (FIG. 2).

Still further, the use the silane based hydrophobic agent enhances the photocatalytic reaction and decreases the generation of undesired NO₂. In a further non-limiting embodiment, the silane based hydrophobic agent can be a fluorinated silane.

In one non-limiting embodiment, a composite ultra-high-performance concrete mixture, comprises cement in an amount between 500 and 680 kg/m³; a photocatalyst in amount between 10 and 30 kg/m³; a recycled glass white nano-silica in amount 40 and 60 kg/m³; calcinated kaolin in amount 70 to 120 kg/m³; a filler in amount between 200 and 300 kg/m³; a fine sand in amount between 600 to 970 kg/m³; and a coarse sand in amount between 400 to 600 kg/m³.

In one non-limiting configuration, the cement comprises cement particles having an average particle size of less than 90 μm.

In another non-limiting configuration, the cement has a specific surface area less than 2.0 m²/g.

In still another non-limiting configuration, the cement has a Blaine fineness of less than 510 m²/kg.

In a further non-limiting configuration, the photocatalyst is in powder form with at least 98% wt Anatase base TiO₂ with B.E.T. surface area between 50 and 400 m²/g.

In a still further non-limiting configuration, the recycled glass white nano-silica has a minimum of 99.0% amorphous SiO₂ content with B.E.T. surface area between 30 and 60m2/g and primary particle size between 30 and 300 nanometers.

In another non-limiting configuration, the calcinated kaolin has particle size between 1 to 7 microns and B.E.T. surface area between 2 to 10 m²/g.

In still another non-limiting configuration, the concrete further comprises Nepheline-Syenite filler, and fine and coarse sand.

In a further non-limiting configuration, the Nepheline-Syenite filler is present in an amount between 200 and 300 kg/m³ and has a particle size less than 75 μm.

In a still further non-limiting configuration, the concrete further comprises Nepheline-Syenite fine sand.

In another non-limiting configuration, the Nepheline-Syenite fine sand is present in an amount between 600 and 970 kg/m³ and has a particle size less than 600 μm.

In still another non-limiting configuration, the concrete further comprises Nepheline-Syenite coarse sand.

In a further non-limiting configuration, the Nepheline-Syenite coarse sand is present in an amount between 400 and 500 kg/m³ and has a particle size less than 1500 μm.

In a still further non-limiting configuration, the concrete further comprises fibers selected from the group consisting of steel fibers, natural fibers, synthetic fibers and mixtures thereof.

In another non-limiting configuration, the concrete further comprises a total water content of between 150 and 275 kg/m³.

In still another non-limiting configuration, the concrete exhibits a compressive strength between 90 MPa and 120 MPa in a normal curing regime.

In a further non-limiting configuration, the concrete exhibits a three bending point flexural strength of between 10and 35 MPa.

In a still further non-limiting configuration, the concrete exhibits a slump flow of between 250 and 350 mm.

In another non-limiting embodiment, a method for manufacturing an enhanced photocatalytic concrete panel, comprises the steps of: sprinkling clear glass and marble aggregates into a urethane resin mold; spreading a first concrete layer over the aggregates; setting an AR-glass mesh on the first concrete layer; spreading a second concrete layer on top of the AR-glass mesh; setting a second AR-glass mesh on the second concrete layer; and spreading a third concrete layer over the second AR-glass mesh to form a composite panel.

In a non-limiting configuration, the clear glass is recycled clear window or municipal waste flint class with aggregate size between 1 and 7 mm.

In another non-limiting configuration, the marble aggregates are natural low dolomite and quartz natural marble with size between 1 and 7 mm.

In still another non-limiting configuration, the clear glass and marble aggregates are mixed in proportion A:B:C: of 1:2.5:2.5 wherein A is white marble size #0 (3.175-1.588 mm); B is clear glass size #0 (3.175-1.588 mm) and C is white marble size #1 (3.175-6.350 mm).

In a further non-limiting configuration, the panel is cured and sealed for 2 days, demolded and further moist cured for 16 to 21 days.

In a still further non-limiting configuration, the panel after secondary curing is sand-blasted (steel shot grit) to expose the concrete matrix and the clear glass and marble aggregates to provide a stone-like appearance.

In another non-limiting configuration, the composite panel, after sand-blasting, is treated with penetrating hydrophobic sealers to decrease photocatalytic NO₂ generation.

In still another non-limiting configuration, the penetrating sealer comprises waterborne flour-silanes and mixtures thereof.

In a further non-limiting configuration, the clear glass aggregates distribute light beneath the panel surface, thereby activating additional photocatalytic particles within the matrix.

In a still further non-limiting configuration, the marble aggregates react with the nitrites' photocatalytic products to further mineralize them and produce soluble CaNO₃ which is then washed off by rain.

In another non-limiting configuration, the composite panel, when exposed to artificial or natural light, reacts with environmental NOx, decreasing its air concentration by at least 35%.

In still another non-limiting configuration, the NOx air concentration is decreased by at least 57%.

In a further non-limiting configuration, the composite panels, upon exposure to bacteria and viruses, is at least 99% effective in killing or stopping their reproduction.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A detailed description of embodiments of the present invention follows, with reference to the attached drawings, wherein:

FIG. 1 is a schematic cross-section taken through a panel according to the invention;

FIG. 2 is an enlarged portion of FIG. 1 showing clear glass and marble interacting with light at a surface of a panel according to the invention;

FIGS. 3 and 4 illustrate a photocatalytic model, and principle of action of panels disclosed herein, respectively;

FIG. 5 illustrates Methylene blue (MBL) degradation results after UV-A radiation of TiO₂ dispersions;

FIG. 6 illustrates Methylene blue (MBL) reaction with TiO₂ dispersion to form L-MBL colorless;

FIG. 7 illustrates Methylene blue (MBL) degradation results after UV-A radiation for different TAKTL SC+ colors (SM-Microseal®);

FIG. 8 illustrates self-cleaning evolution of TAKTL SC+ panel (WH87 Microseal®) during simulated environmental conditions;

FIG. 9 illustrates a photocatalytic reactor used for NOx abatement studies (courtesy TU/e, the Netherlands);

FIG. 10 illustrates results obtained according to ISO 22197-1, a) W15 WH87 REF (half media-blast), b) TAKTL SC+ W15 WH87 (half media-blast);

FIG. 11 illustrates results obtained according to modified ISO 22197-1, a) W15 WH87 REF (half media-blast), b) TAKTL SC+ W15 WH87 (half media-blast), panel tested after water wash to simulate building rain;

FIGS. 12a-12c illustrate results obtained according to modified ISO 22197-1 [19], a) W10 WH87 SC+ (half media-blast), b) TAKTL SC+ W10 WH87 (half media-blast) Microseal, c) W10 WH87Korsa SC+ (glass and marble face aggregates) Microseal;

FIGS. 13a and 13b illustrate the mechanism of photocatalytic inactivation of bacteria (FIG. 13a) and viruses (FIG. 13b) by reactive oxygen species (ROS);

FIGS. 14a-14c illustrate ISO 18061 test on SC+ samples, surface inoculation (FIG. 14a), samples covered (FIG. 14b), UV radiation box (FIG. 14c);

FIGS. 15a-15c illustrate Moss growth test, mixing buttermilk and moss spores (FIG. 15a), surface painting (FIG. 15b), samples storage (FIG. 15c);

FIG. 16 illustrates moss growth test surface comparison, a) all samples after 15 months, b) SA72 samples at beginning of test, c) SA72 samples after 15 months;

FIG. 17 illustrates an accelerated weathering color change (ΔE) after 500-h of continuous light exposure (dry and wet) according to ASTM G155 and ASTM D2244;

FIG. 18 illustrates accelerated weathering color parameter (ΔE, Δa and Δb) changed after 500-h of continuous light exposure (dry and wet) according to ASTM G155 and ASTM D2244, a), c), d) rough 1 finish, b), d), f) rough 1 media-blast finish);

FIG. 19 illustrates accelerated weathering color parameter (ΔE, Δa and Δb) changed after 500-h of continuous light exposure (dry and wet) according to ASTM G155 and ASTM D2244, a), c) rough 1 finish, b), d) rough 1 media-blast finish);

FIG. 20 illustrates natural outdoor weathering color change (ΔE) after 9 months, measured according to ASTM D2244;

FIG. 21 illustrates natural outdoor weathering color parameter (ΔE, Δa and Δb) change after 9 months, a), c) rough 1finish, b), d) rough 1 mediablast finish); and

FIG. 22 illustrates a natural outdoor weathering color parameter (ΔE, Δa and Δb) change after 9 months, a), c) rough 1finish, b), d) rough 1 mediablast finish).

### DETAILED DESCRIPTION

The invention relates to a concrete composition and, more particularly, to a UHPC concrete composition containing photocatalyst and recycled glass or ceramic materials such as glass white nano-silica. The recycled glass helps to keep carbonation low in the concrete, and this helps to maintain the activity of the photocatalyst.

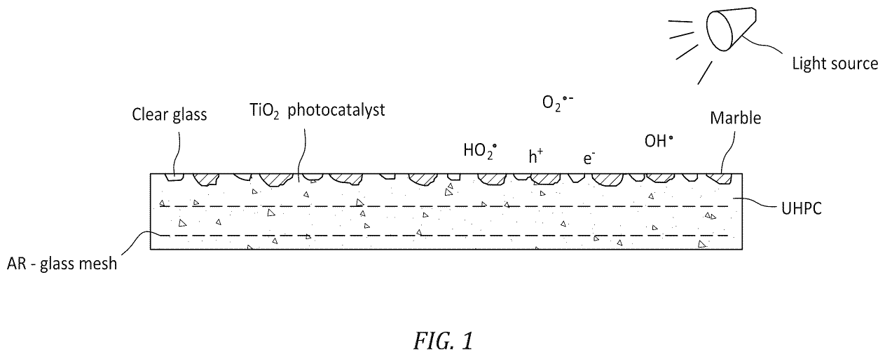

FIG. 1 shows a panel which can be formed from a UHPC concrete material and can include a photocatalyst such as TiO₂. An outer facing surface of the panel includes clear glass and marble components as shown in FIG. 1. These components help to spread light into the body of the panel to help expose more of the photocatalyst of the panel and thereby provide more useful effects from the panel.

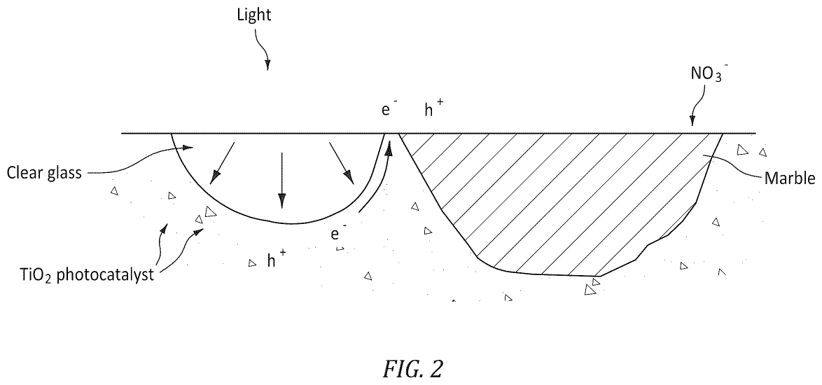

FIG. 2 shows an enlarged portion of a surface of the panel of FIG. 1, and shows light encountering a clear glass particle at the surface of the panel, and then passing through the clear glass to contact the underlying concrete including the photocatalyst distributed therein.

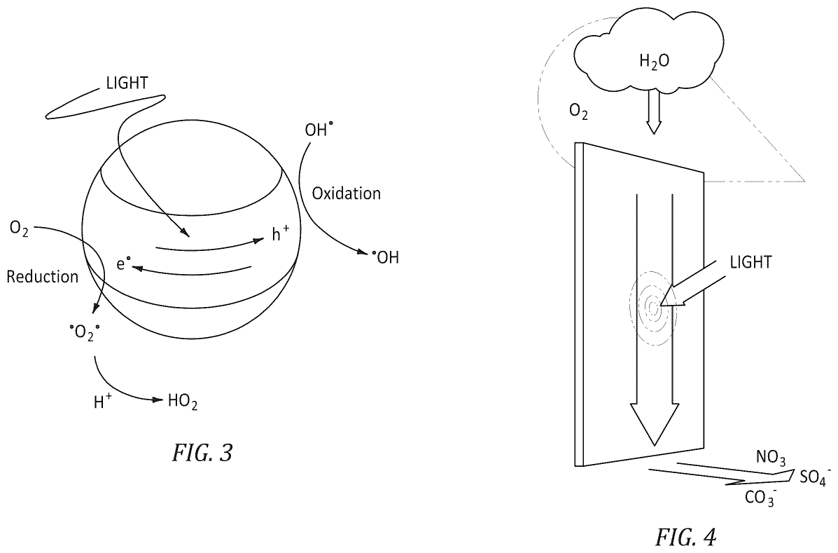

FIGS. 3 and 4 further illustrate the reactions that take place. Concrete panels as disclosed herein can be permanently self-cleaning, pollution-removing, and antimicrobial. The photocatalytic multifunctional properties of the inventive panels are activated by natural or artificial light to break down organic and inorganic contaminants and other environmental toxins. Photocatalytic action begins when titanium-based nanoparticles, integral to the UHPC mix, are activated by the energy (hv) of the light source.

The energy of the light produces electron (e⁻)-hole (h⁺) pairs:

$$T i O 2 + hv \to {h}^{+} + {e}^{-} \qquad Eq . 1$$

The h⁺ reacts with OH⁻ to dissociate from environmental water and form hydroxyl radicals OH^{·}:

$${h}^{+} + {OH}^{-} \to {OH}^{} \qquad Eq . 2$$

The e-reacts with molecular oxygen (O₂) to form the superoxide anion O₂·⁻:

$${e}^{-} + {O}_{2} \to {O}_{2}^{-} \qquad Eq . 3$$

Afterwards, the superoxide anion further reacts with H⁺ dissociated from water to produce HO₂^{·} radicals:

$$H + + {O}_{2}^{-} \to H {O}_{2}^{} \qquad Eq . 4$$

This process of oxidation and reduction of the TiO₂ nanoparticles together with the other chemical components of the UHPC formulation work to decompose pollutants on and near the panel surface, see FIG. 3. When activated, environmental humidity breaks down pollutants (i.e., NOₓ, SOₓ, CO₂, etc.) into harmless soluble solids that are washed away by rain. These oxidative and reductive effects are also responsible for antibacterial and antiviral properties of the disclosed panels.

The composition of the UHPC material is useful in providing extended catalytic activity of the photocatalyst. Table 1 below illustrates properties of the composition according to one non-limiting embodiment:

TABLE 1
Composition of UHPPC matrix

| | Mix design |
|---|---|
| Name | (Kg/m³) |
| White Cement | 500-680 |
| Recycled glass white nano-silica | 40-60 |
| Calcinated Kaolin | 70-120 |
| Nepheline-Syenite (filler) | 200-300 |
| Nepheline-Syenite (Medium) | 600-970 |
| Nepheline-Syenite (Coarse) | 400-600 |
| Photocatalytic TiO₂ Anatase | 10-30 |
| (Tronox PC105) | |

Each of the materials of Table 1 has characteristics that are also useful in connection with the desired results obtain according to the invention. These characteristics or properties are shown in Table 2 below:

TABLE 2
Raw material characteristics (granular materials)

| | | Specific | | Particles |
|---|---|---|---|---|
| | | surface | Mean | parameters |
| | Density | area | particle | [D 10/D 50/D 90] |
| Name | (g/cm³) | (m²/g) | size (μm) | (μm) |
| White Cement | 3.13 | 0.74-1.47 | 9.8-20.4 | 0.8/11.1/34.3 |
| Recycled | 2.10-2.20 | 30-60* | 0.2-0.3 | 0.10/0.24/0.60 |
| glass white | | | (30-300 nm) ⁺ | |
| nano-silica | | | | |
| Metakaolin | 2.50 | 0.93-1.27 | 5.1-6.6 | 1.0/3.9/12.9 |
| Nepheline- | 2.61 | 0.48-0.70 | 16.4-25.8 | 1.7/14.6/49.7 |
| Syenite | | | | |
| (filler) | | | | |
| Nepheline- | 2.61 | 0.01-0.03 | 256.3-313.2 | 121.9/273.5/467.0 |
| Syenite | | | | |
| (fine) | | | | |
| Nepheline- | 2.61 | 0.002-0.004 | 1065-1143 | 724.5/1072/1607 |
| Syenite | | | | |
| (Coarse) | | | | |
| Tronox PC105 | 3.9-4.1 | 80-100* | 0.017 | — |

*BET surface area, ⁺ primary particles

Five (5) different mixes of concrete were prepared as shown in Table 3 below:

TABLE 3
Composition of different UHPC mixes

| | UHPC Mixes | | | | | |
|---|---|---|---|---|---|---|
| | Mix A | Mix B | Mix C | Mix D | Mix E | |
| Material | Amount (Kg/cm³) | | | | | |
| White Cement | 600 | 600 | 650 | 650 | 650 | |
| White pigment | 26 | 26 | 35 | 35 | 35 | |
| (TiO₂ rutile) | | | | | | |
| Photocatalyst Tronox | 0 | 40 | 26 | 26 | 26 | |
| PC105 | | | | | | |
| Recycled glass white | 48 | 48 | 52 | 52 | 52 | |
| nano-silica | | | | | | |
| Metakaolin | 104 | 80 | 87 | 87 | 87 | |
| Nepheline-Syenite | 202 | 202 | 298 | 298 | 298 | |
| (filler) | | | | | | |
| Nepheline-Syenite | 691 | 691 | 963 | 963 | 963 | |
| (fine) | | | | | | |
| Nepheline-Syenite | 479 | 479 | — | 479 | 479 | |
| (Coarse) | | | | | | |
| AR-glass strands | 41 | 41 | 41 | 41 | 41 | |
| (12 mm) | | | | | | |
| Superplasticizer | 28 | 28 | 33 | 33 | 33 | |
| (PCE-type) | | | | | | |
| Water | 210 | 210 | 226 | 226 | 226 | |
| Water/binder | 0.287 | 0.287 | 0.296 | 0.296 | 0.296 | |
| Water/solid | 0.105 | 0.104 | 0.116 | 0.116 | 0.116 | |
| White marble | 0 | 0 | 0 | 0 | 1 | part |
| aggregates size # 0 | | | | | | |
| Clear glass | 0 | 0 | 0 | 0 | 2.5 | part |
| aggregates size # 0 | | | | | | |
| White marble | 0 | 0 | 0 | 0 | 2.5 | parts |
| aggregates size # 1 | | | | | | |
| Fluro-silane sealer | 0 | 0 | 0 | 44 g/ft2 | 44 | g/ft2 |

Of these mixes, Mix E is made according to the present invention, while Mixes A-D are prepared for comparison purposes. Furthermore, based upon the results of this testing, a combination of Mix B with aggregates is also considered a composition according to the invention and provides good NOx reduction after washing.

Mixes A-E were subjected to modified ISO 22197-1 NOx degradation standard testing, and the results are shown below in Table 4:

TABLE 4
Summary results according to modified ISO 22197-1 NOx degradation standard test.

| Mix | Mix A | Mix A | Mix B | Mix B | Mix C | Mix D | Mix E |
|---|---|---|---|---|---|---|---|
| Finish (sand blasting | 50% | 50% | 50% | 50% | 50% | 50% | 100% |
| level) | | | | | | | |
| Sealer | none | none | None | none | none | yes | yes |
| NOx flow rate (L/min) | 3.0 | 1.5 | 3.0 | 1.5 | 1.5 | 1.5 | 1.5 |
| NOx concentration (ppmv) | 1.0 | 0.3 | 1.0 | 0.3 | 0.3 | 0.3 | 0.3 |
| Humidity (%) | 50 | 50 | 50 | 50 | 50 | 50 | 50 |
| UV light intensity (W/m²) | 10 | 10 | 10 | 10 | 10 | 10 | 10 |
| NO removed (%) | 11.3 | 34.8 | 54.7 | 97.4 | 61.5 | 38.3 | 64.5 |
| Selectivity (% NO₂ | 3.8 | 11.6 | 19.8 | 6.4 | 9.3 | 4.9 | 7.2 |
| generated) | | | | | | | |
| NOₓ removed (%) | 7.5 | 23.2 | 34.9 | 91.0 | 52.2 | 33.4 | 57.3 |
| Water washed | No | Yes* | No | Yes* | No | No | No |

*retest after water washing and drying.

Further testing was conducted for effectiveness at destroying coronavirus. Table 5 sets forth the results:

TABLE 5
Summary results according to modified ISO 18061-2014 for coronavirus
surrogates 229E (HCoV-229E) and OC43 (HCoV-OC43)

| | | | | | | Percentage |
|---|---|---|---|---|---|---|
| | | | Infectivity | UV | UV | virus |
| Sam- | | Cell | Titer | Intensity | radiation | reduction |
| ple | Virus | line | TCID50/ml | (mW/cm2) | time (h) | (killed)* |
| 1 | 229E | MRC-5 | 10⁵ | 0.25 | 4 | 100% |
| 2 | 229E | MRC-5 | 10⁵ | 0.25 | 4 | 99% |
| 3 | 229E | MRC-5 | 10⁵ | 0.25 | 4 | 100% |
| 4 | OC43 | HCT-8 | 10⁵ | 0.25 | 4 | 100% |
| 5 | OC43 | HCT-8 | 10⁵ | 0.25 | 4 | 100% |
| 6 | OC43 | HCT-8 | 10⁵ | 0.25 | 4 | 100% |

*Percentage reduction calculated per original infectivity titer inoculum

The composition tested was Mix B from Table 3 set forth above. These results are expected to carry forward as well for Mix E. As shown, the concrete composition as tested was highly effective at reducing or killing the tested cell lines of coronavirus.

Further evaluation was conducted to test TiO₂ photocatalytic verification and self-cleaning according to ISO 10678. The first issue to address was evaluation and selection of the TiO₂ nanoparticles to use.

In the initial stage of development, a range of commercially available TiO₂ nanoparticles was selected to evaluate for intrinsic photocatalytic properties using the ISO 10678-10 modified standard [2]. This standard is used in fine and advanced ceramics to determine the photocatalytic activity of surfaces by the degradation of the dye molecule methylene blue (MBL) in aqueous solution using artificial ultraviolet (UV-A) radiation and to characterize the ability of photoactive surfaces or powders to degrade dissolved organic molecules under ultraviolet light wave lengths. The test method is also applicable to evaluation of the specific self-cleaning photoactivity of surfaces.

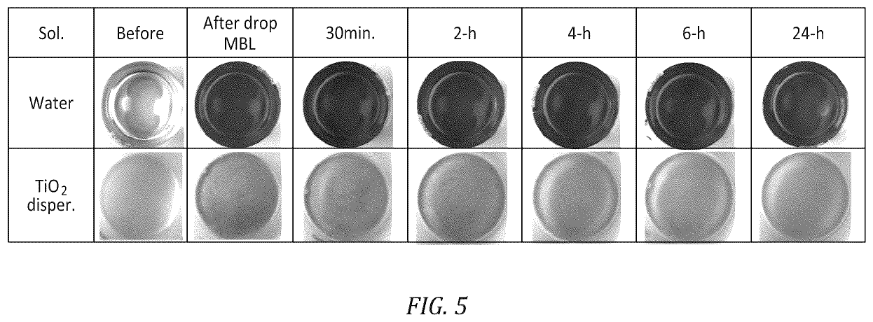

To verify and rank the photocatalytic activity of the TiO₂ powders selected for this test series, samples with 10% wt. dispersion of the particles in water were prepared by adding 10 g of TiO₂ powder to 90 g distilled water (DI) in a glass beaker. The dispersion was stirred for 5 min. Then 50 ml of the prepared dispersion was mixed with one drop of 0.1 mM of MBL solution again stirring for 5 min. The dyed solution was in a dark box containing a UV-A light (Waveform Lighting's real UV™/Wavelength 365 nm/Irradiance 650 μW/cm²/Power 20 W) and irradiated for 30 min and 2, 4, 6 and 24 hours. After each exposure the color change of the solution was visually monitored and compared with a reference sample composed only of DI water and the same initial concentration of MBL. The results of the TiO₂ powder with the highest-ranking photocatalytic activity are displayed on FIG. 5.

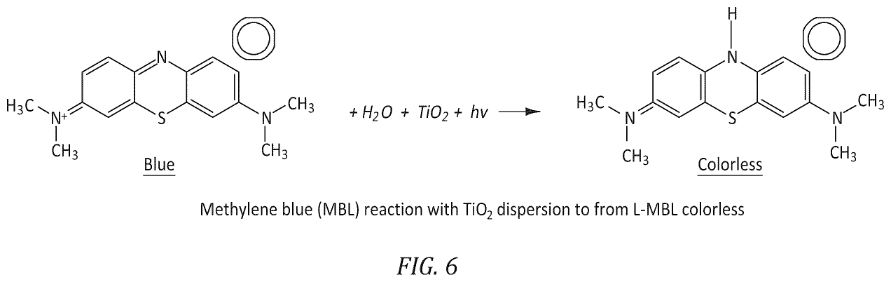

Methylene blue (MBL) or tetramethyl-thionine chloride, (C₁₆H₁₈ClN₃S) is a heterocyclic aromatic dye that is brightly blue colored in an oxidizing environment, see FIG. 6.

Because of the photocatalytic action of TiO₂ powder in the presence of light, it changes upon reduction to leuco-methylene blue (L-MBL), which is colorless. This change in color is considered visual evidence of the degradation of organic molecules and serves as a test model pollutant. As is shown in FIG. 5, 24 hours of exposure resulted in an almost complete color change of the TiO₂ dyed dispersion, indicating a high degree of MBL degradation and photocatalytic activity.

In FIG. 5 it is also evident that the degradation of MBL is slower for the first 30 minutes, becoming more accelerated after 2-h of exposure. After 24 hours, it is expected that the MBL will be fully degraded into the final products SO₄⁻², CO₂, NH₄⁺ and NO₃⁻ as previously reported by several researchers and further evidenced in this test series with a complete color change to clear. The results obtained in this series demonstrate the high photocatalytic activity of the TiO₂ powder selected for the formulation of panels as disclosed herein.

### Self-Cleaning Evaluation of SC+ Panels

The A|UHPC mix designs were formulated and adjusted to incorporate the selected TiO₂ photocatalytic powder to produce panels. These panels, produced in a smooth finish and different colors (WH87, BO78, TI63, PL75, RED and BLACK) were used to evaluate the self-cleaning and organic decomposition using MBL. The methodology used was based on the ISO 10678 modified method with the use of a container glued to the surface of 2×2 in. panel sections (with Microseal®). The clear containers (sealed Petri dishes) were filled with DI water containing MBL as the model pollutant (0.1 mM). The panels later were irradiated for 3-h, 6-h, 1-d, 3-d and 6-d to determine the photocatalytic degradation induced by the surface of the panels. As in the previous test series, a dark box containing a UV-A light (Waveform Lighting's real UV™/Wavelength 365 nm/Irradiance 650 μW/cm²/Power 20 W) was used for the experiment.

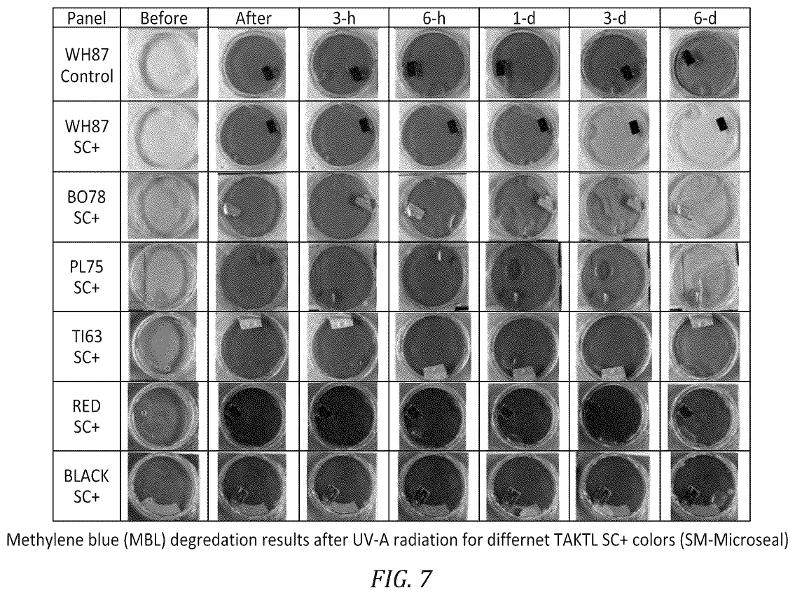

The visual results obtained for the self-cleaning effects of the various TAKTL SC+ colored panels tested are displayed in FIG. 7. In this figure, it is possible to observe the difference between a conventional panel (Color WH87/Texture Smooth/Finish Microseal) and a panel as disclosed herein but of the same color, texture, and finish. It is noticeable that after 6-d of exposure the model pollutant (MBL) was completely degraded, with the degradation rate slower during the initial 1-d and accelerated after 3-d of exposure. Compared with the pure photocatalytic powder experiment, the degradation of the model pollutant is slower, as expected with the lower relative concentration of integrated photocatalytic powder in the A|UHPC mix design.

Similarly, pigmented panels made with standard colors (WH87, BO78, PL75, TI63) and two custom colors with high pigment loads (RED and BLACK), exhibited progressive photocatalytic degradation of the MBL until 6-d. The main difference observed is that the darker the panel and higher the pigment load, the slower the photocatalytic rate as compared to the present invention (SC+ WH87 (white)), which exhibited the highest efficiency. This behavior is expected as it has been demonstrated that iron-based concrete pigments used to formulate colored concrete (Fe₂O₃, Fe₃O₄ and FeOOH) present their own catalytic activity which is interacting with the electron-hole pair formed (Eq. 1 to 4) as a result of the photocatalytic reaction, which are later liberated to produce self-cleaning effects at a slower rate. By contrast, when conventional rutile-based white pigment (case WH87) is mixed with photocatalytic TiO₂ nanoparticles, an enhanced reaction rate and self-cleaning effect have been reported. In summary, this experiment demonstrated the self-cleaning properties of the present invention as embodied in a proposed SC+ product line, including dark saturated colors, with the more highly pigmented colors exhibiting a slower self-cleaning ability that will require more exposure time to completely degrade pollutants.

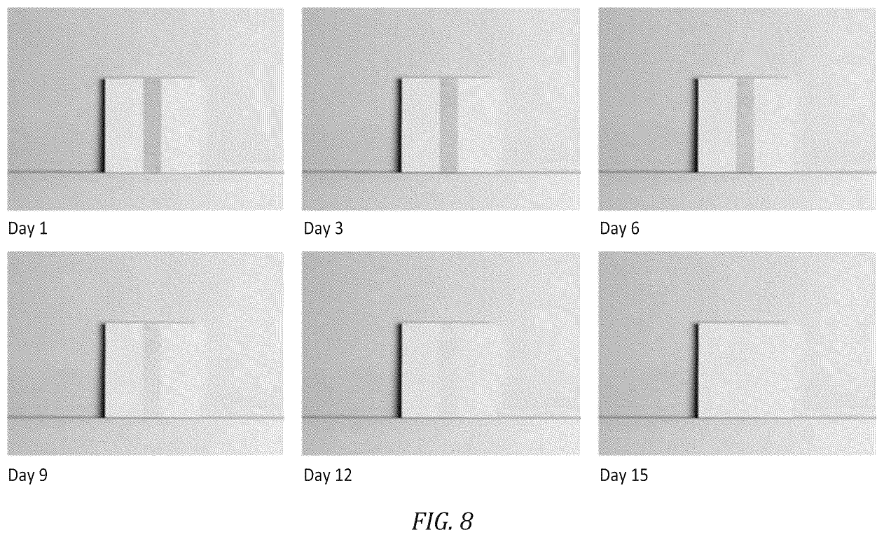

As in real conditions, façade panels are not going to be immersed in water as specified in the ISO test. Therefore, a modification of the ISO 10678 procedure was performed to evaluate the self-cleaning effects on inventive compositions in conditions more closely matching natural exposure. For this, a SC+ WH87 panel was tinted with a photocatalytic indicator ink (FIG. 8), inserted in the same black box for several days and exposed to UV-A radiation for a period of 8-h each day. Each day, the panel was extracted for visual inspection and record. In addition, before inserting each panel back into the box, a light water spray was applied to simulate environmental moisture conditions. This procedure was repeated until a complete self-cleaning effect of the surface was achieved.

In FIG. 8 it is possible to observe that panels as disclosed herein under conditions closer to reality took approximately 15-d to completely degrade the model pollutant ink. This is clear evidence of the self-cleaning ability of the panel surface. The degradation of the model pollutant represents the inventive panel's response to a wide variety of common particulates including nitrogen oxide, soot, grease, VOCs, and microbes as will be demonstrated in other sections of this disclosure.

### Degradation of Air Polutants (ISO 22197-1)

A variety of air pollutants are known or suspected to have harmful effects on human health and the environment. In most areas of United States, these pollutants are principally the products of combustion from space heating, power generation or from motor vehicle traffic. The primary pollutants emitted by vehicles are carbon monoxide (CO₂), oxides of nitrogen (NOₓ), sulphates (SOₓ), volatile organic compounds (VOCs) and particulates. These pollutants have an increasing impact on outdoor and indoor air quality. In addition, photochemical reactions resulting from the action of sunlight on NO₂ and VOCs lead to the formation of ozone, a secondary long-range pollutant, with impacts in rural areas often far from the original emission site. Acid rain is another long-range pollutant influenced by vehicle NOₓ emissions and resulting from the transport of NOₓ, oxidation in the air into NO₃, and subsequent precipitation of nitrogen acid, resulting in harmful consequences for building materials (corrosion of the surface) and vegetation. A solution for the air pollution from traffic can be found in the treatment of pollutants as close to the source as possible. Therefore, several researchers have been adding photocatalytic materials to the surface of pavement and building materials. In combination with light, the pollutants are oxidized, due to the presence of the photocatalyst and precipitated on the surface of the material. Consequently, they are removed from the surface by rain. In this context, the presently disclosed material and panels made therefrom were developed intentionally to be used as a building facade panel to provide abatement of air pollutants and improve air quality. Different standard methods have been developed to evaluate the degradation of gaseous air pollutants as NOₓ and VOCs (also called BTEX for Benzene, Toluene, Ethylbenzene and Xylene) using photocatalytic materials.

To evaluate the potential air purification of the present invention (TAKTL SC+) the ISO 22197-1 was selected. To determine the air purifying activity of TAKTL SC+ the oxidation of NO and NO₂ into NO₃ is determined. Emphasis is put on this pollutant, since it is one of the most important pollutants produced by traffic and plays a major role in the formation of smog and ozone. The oxidation of the NOₓ in the presence of TiO₂ nanoparticles and light (hv) is simplified in the following equations:

$$N O + O {H}^{} \to N {O}_{2} + {H}^{+} \qquad Eq . 5$$

$$N {O}_{2} + O {H}^{} \to N {O}_{3}^{-} + {H}^{+} \qquad Eq . 6$$

The NO₃ formed during this process precipitates on the surface of the TAKTL SC+ panel and is later washed out by the rain or mineralized in the surface of the panel.

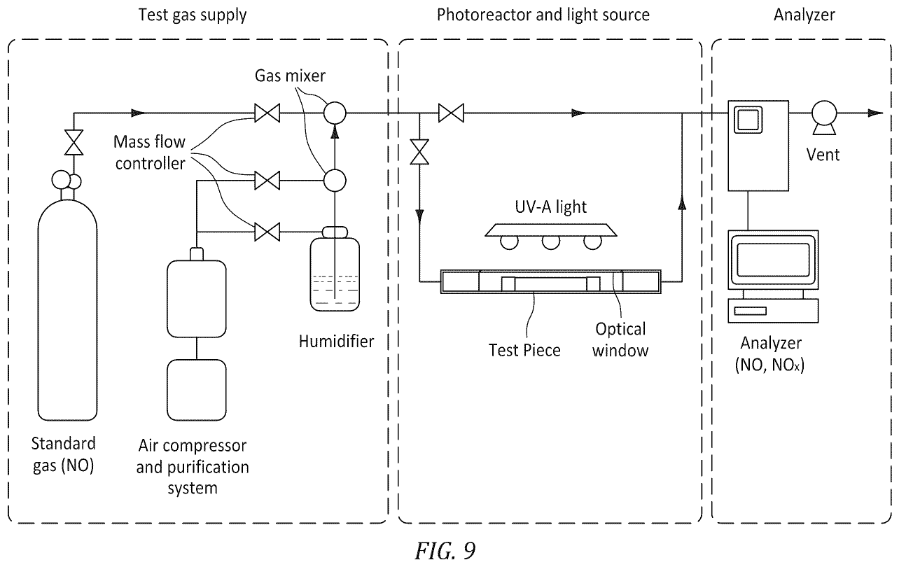

As mentioned above, the capacity of TAKTL SC+ panels to remove air pollutants was assessed by using nitric oxide (NOₓ) as a model pollutant. A plug-flow experimental setup located at Eindhoven University of Technology in The Netherlands (TU/e) was used in this study for evaluating photocatalytic efficiency (FIG. 9).

Nitric oxide (NOₓ) was mixed with a synthetic air and adjusted to the desired concentration (1 ppmv) and flow rate (3 L/min) by the mass control meters. The applied light source was composed of three fluorescent tubes of 25 W each, emitting high-concentrated UV-A radiation in the range of 300-400 nm.

The experimental conditions, such as the pollutant concentration, flow rate, humidity, and light intensity (irradiance 10 W/m²) were fully controlled and monitored. The temperature (69 F) and the humidity (50%) were measured at the inlet of the reactor. The outlet concentration of NO and NO₂ were measured and interpreted as the NOₓ concentration. The pollutant concentration was measured by an online NOₓ analyzer APNA-370(Horiba). The APNA-370 continuously monitors the NOx concentration using a crossflow modulated semi decompression chemiluminescence method. The concentration measurement was performed automatically every 5 seconds with a sampling flow rate of 0.8 L/min.

Different reference TAKTL Standard formulations and various TAKTL SC+ panels with and without Microseal (fluorinated silane) were evaluated. The active sample surface size of the panels was fixed as 190×87 mm (7.5×3.5 in.).

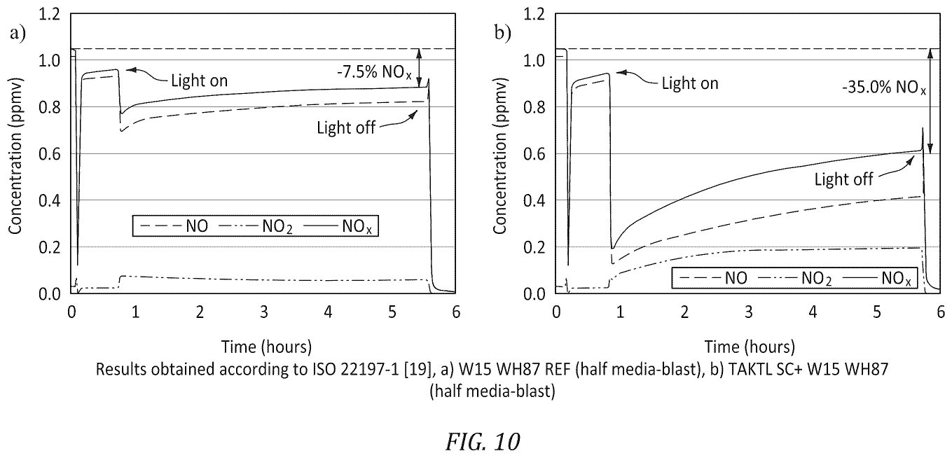

The results of NOₓ degradation following the standard condition recommended by ISO 22197-1 are displayed in FIG. 10.

As can be seen in FIG. 10, reference TAKTL panels presented a small NOₓ degradation characterized by an initial gas absorption at the first 30 min on the dark (due to surface micro-porosity). Once the light is on, a small degradation of NOx started to appear of approximately 7.5% at the end of the test. The reason of this is that normal Rutile (Ti) based white pigments contain small impurities of crystalline phases that present some degree of photocatalytic activity (Ti-Anatase). In the case of TAKTL SC+, a high NOₓ degradation is observed immediately when the light is turned on. The degradation rate decreased over time during the test until an asymptotic degradation degree of 35% was reached. The decrease in NOₓ abatement with time is related to the quick saturation of photocatalytic sites on the surface (selectivity), resulting in lower mineralization and formation of NO₃⁻, which is evident by the increase of intermediate NO₂ generation. This result of higher NO₂ and lower NO₃⁻ concentration is also the consequence of the very high concentration of NOₓ related to the exposed surface area prescribed by the ISO standard, which is one of the drawbacks of using the ISO 22197-1 standard.

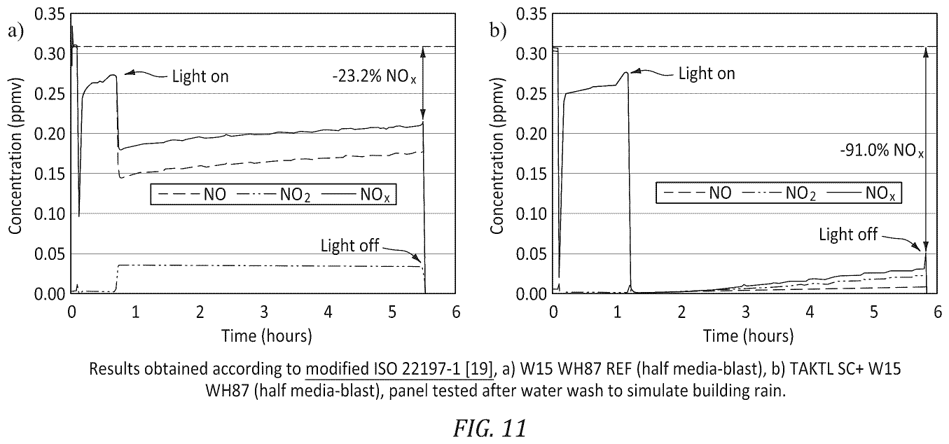

Therefore, to further assess the performance of SC+ panels under more realistic conditions of lower gas flow rate (1.5 L/min) and lower pollutant concentration (0.3 ppmv), the same panels used in the initial experiment were evaluated after being washed with water to regenerate the photocatalytic activity (simulated rain wash). As can be seen in FIG. 11, the NOₓ degradation for the standard panel (W15), representing Mix A, increased from 7% to 23% with decreased NO₂ amounts (Table 6). This means that lower gas rate and concentration allows for more degradation by not completely saturating the photocatalytic sites of the surface. As expected, the selectivity decreased as more NO₂ was generated. In contrast, the TAKTL SC+ panel (Mix-B) showed a surprisingly high degradation rate (91%) and lower generation of intermediate NO₂ (selectivity). This is the result of surface regeneration from water washing and the presence of the specific photocatalytic nanoparticles on SC+ panels. It is important to note that EPA defines the concentration of NOx 1-h exposure around 0.10 ppmv and the average ambient concentration in U.S. around 0.05 ppmv. Furthermore, the NOₓ concentration close to highways and busy cities street is around 0.02 to 0.30 ppmv, similar to these modified test conditions, leading to the conclusion that higher air cleaning purification is expected by SC+ panels in real conditions.

TABLE 6
Summary results obtained according to standard and
modified ISO 22197-1 [19] NOₓ degradation.

| | sample | | | | | | |
|---|---|---|---|---|---|---|---|
| | | | | W15 | | | |
| | | W15 | W15 | SC+ | W10 | W10 | W10 |
| Name | W15 | (w) | SC+ | (w) | SC+ | SC+ | SC+ |
| Finish | SM/MB | SM/MB | SM/MB | SM/MB | SM/MB | SM/MB | MB + AGG |
| Sealer | None | None | None | None | None | Microseal | Microseal |
| Flow rate (L/min) | 3 | 1.5 | 3 | 1.5 | 1.5 | 1.5 | 1.5 |
| NOₓ Conc. (ppmv) | 1.0 | 0.3 | 1.0 | 0.3 | 0.3 | 0.3 | 0.3 |
| Humidity (%) | 50 | 50 | 50 | 50 | 50 | 50 | 50 |
| UV Light Int. | 10 | 10 | 10 | 10 | 10 | 10 | 10 |
| (W/m²) | | | | | | | |
| NO Removed (%) | 11.3 | 34.8 | 54.7 | 97.4 | 61.5 | 38.3 | 64.5 |
| Selectivity (% NO₂ | 3.8 | 11.6 | 19.8 | 6.4 | 9.3 | 4.9 | 7.2 |
| gen.) | | | | | | | |
| NOₓ removed (%) | 7.5 | 23.2 | 34.9 | 91.0 | 52.2 | 33.4 | 57.3 |

(W): water washed, SM: smooth, BM: Media-blast, ppmv: parts per million in volume, AGG: phase glass and marble aggregates (KORSA ®).

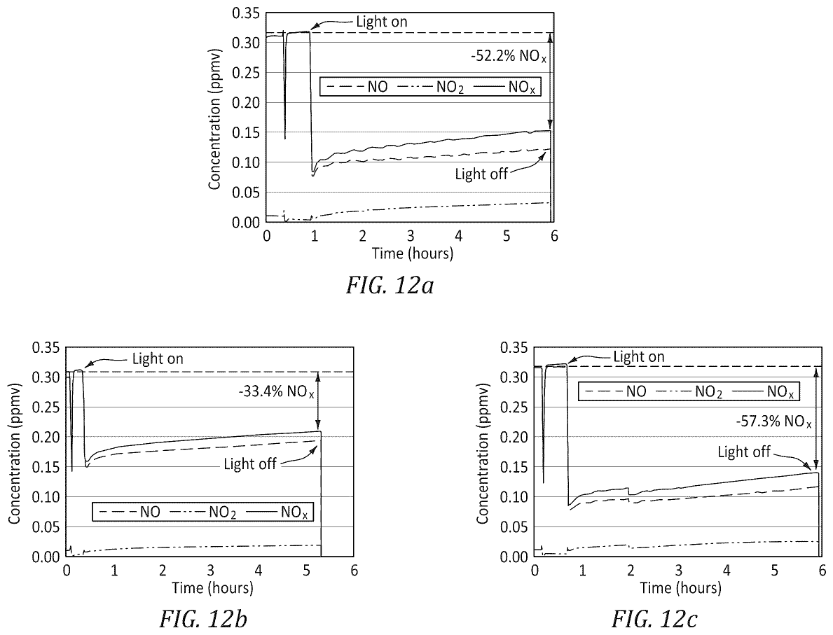

Another evaluation was performed to determine the effect on NOₓ degradation of incorporating either an anti-graffiti coating (Microseal) or face aggregates in the manufacture of SC+ panels. The results are shown on Table 6 and FIGS. 12a-12c. From FIG. 12a it is possible to observe that W10SC+ panels (Mix-C) tested in conditions close to real concentrations of NOₓ displayed a degradation of around 52% with a relative medium selectivity. The higher degradation is possibly caused of the use of finer micro-aggregates in the Mix-C compared with Mix-A. Interesting results were obtained on Mix-D panels finished with anti-graffiti Microseal coatings, in which the NOₓ degradation is decreased to 33%, possibly caused by the relative decrease in water absorption of the surface due to the hydrophobic nature of the sealer. Nevertheless, the selectivity or generation of NO₂ was minimized, which demonstrates that SC+ panels are less prone to deactivation by surface saturation of NO₃⁻. Finally, SC+ Korsa panels (Mix-E) were tested with clear glass and marble aggregates together with a Microseal finish. In this case, an enhancement of the degradation rate of NOₓ was observed together with a low selectivity. It is believed, based on existing reports, that the increase in efficiency of Mix-E Microseal panels are the results of higher light dispersion due to the use of clear glass (further light penetration of the bulk of the material) and the better mineralization of NO₃⁻ on the surface of marble aggregate [formation of Ca(NO₃)₂].

Other parameters which are important for these reactions and the efficiency of SC+ panels are the relative humidity and the temperature. At higher temperatures, the conversion will be better.

Relative humidity is important since the water in the atmosphere plays a role in the adhesion of the pollutants at the surface and therefore also the conversion rate. In the case of a higher relative humidity, the conversion will be lower. Optimal conditions would therefore be reached on hot summer days with high temperatures and low relative humidity. It is also on those days that the risk of smog during the summer is the highest and thus the efficiency of the air purification is the highest. As demonstrated, to retain the efficiency of the panels, the deposits of NO₃⁻ will have to be washed away by rain or by cleaning the surface with water at intervals (in the case of indoor applications).

Based on the results presented, it is possible to conclude that TAKTL SC+ will present a minimum NOx degradation of 35% which could be higher depending on the panel finish and the environmental condition of the installation sites. In addition, due to the high photocatalytic activity observed, it can be inferred that other air pollutants would be similarly degraded. Substances that can be abated by photocatalysis based on the literature are:
Inorganic compounds: NOₓ, SOₓ, CO, NH₃, CH₃S and H₂S.
Chlorinated organic compounds: CH₂Cl₂, CHCl₃, CCl₄, 1,1-C₂H₄Cl₂, 1,2-C₂H₄Cl₂, 1,1,1-C₂H₃Cl₃, 1,1,2-C₂H₃Cl₃, 2-C₂H₂Cl₄ and the like, C₂HCl₃; C₂Cl₄, dioxins, chlorobenzene, chlorophenol, etc.
Organic compounds: CH₃OH, C₂H₅OH, CH₃COOH, CH₄, C₂H₆, C₃H₈, C₂H₄, C₃H₆, C₆H₆, phenol, toluene, ethylbenzene, xylene, phenanthroquinone and others VOCs which are tested according to ISO 22197-2 to ISO 22197-5.
Pesticides: Triadimefon, Pirimicarb, Asulam, Diazinon, MPMC; atrazine, etc.

Other compounds would be bacteria, viruses, moss, mold, etc., as will be demonstrated in the below.

### Antimicrobial Properties (ISO 18061 and Moss Growth)

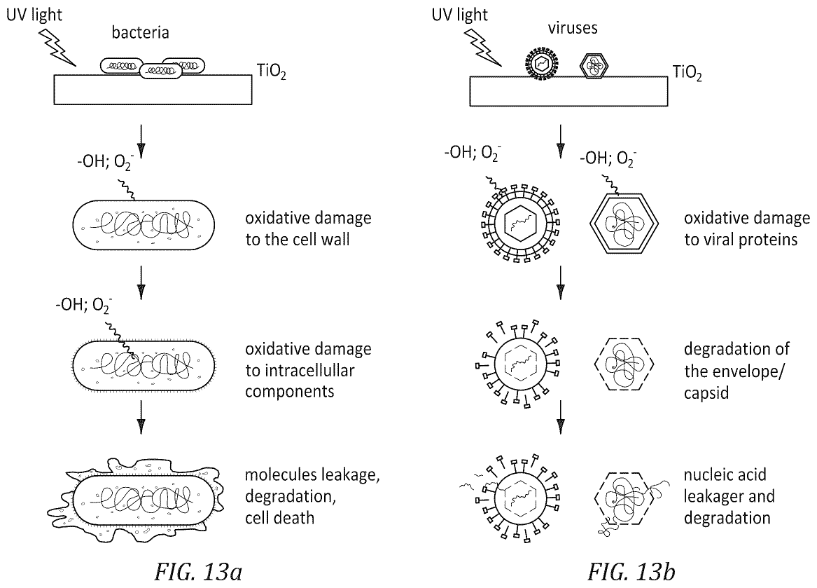

Growing concerns for human health and quality of life have led to the implementation of nanoparticle photocatalysts in civil structures with fabricated self-disinfecting surfaces, primarily for public places that need a high level of hygiene, such as in hospitals, schools, public transportation, and so on. It has been proven that a semiconductor with photocatalytic properties such as TiO₂ provides surfaces with self-disinfection mainly due to the generation of oxygen reactive species (O₂·⁻, HO^{·}, HO₂^{·}, H₂O₂, etc.) that interact with microorganism cells (like virus, bacteria, mold, etc.) breaking them down and decreasing their growing rate and survival. FIGS. 13a and 13b show an example on how this mechanism works.

### TAKTL SC+ Panel Antiviral Evaluation

To determine the antiviral effects on TAKTL SC+, the ISO 18061-2014 standard was used. This test method is used for fine ceramics to determine the antiviral activity of semiconducting photocatalytic materials using bacteriophage Q-beta, which is a positive-strand RNA virus that infects bacteria like Escherichia coli. The tests were performed by the Tile Council of North America (TCNA) laboratory, employing a modified method to determine the effects of SC+ on human coronavirus surrogates 229E and OC43. Human coronavirus 229E is a species of coronavirus which infects humans and bats. It is an enveloped, positive-sense, single-stranded RNA virus which enters its host cell by binding to the APN receptor. Along with Human coronavirus OC43, 229E is a member of the genus Alphacoronavirus and subgenus Duvinacovirus and are structurally like SAR-COVID-19 virus, making them useful surrogates for this virus.

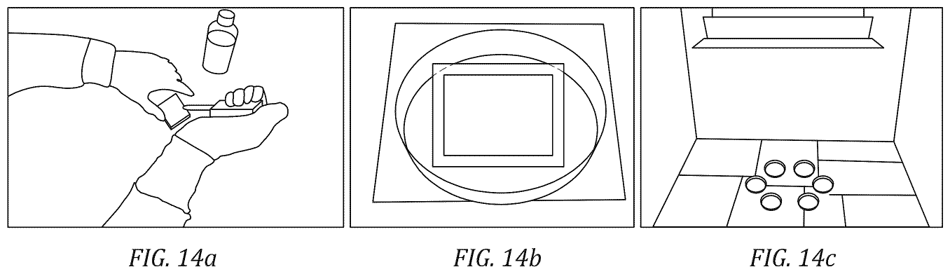

For this evaluation, ten 2×2 in., W15 SC+ smooth WH87 samples (Mix-B) were tested for antiviral determination. In this test, SC+ samples were inoculated with 0.15 ml of virus suspension (FIG. 14a), covered with glass (FIG. 14b), and exposed to a UV-A radiation of 0.25 mW/cm² for a period of 4-h (FIG. 14c).

Test conditions were 25+/−1° C. in temperature a 90% of humidity. Samples from panel surfaces after irradiation were recovered to produce agar virus supports that were incubated and tittered for virus infectivity with 10⁵ TCID50/ml solution and finally compared with control samples to determine the percentage reduction in viral charge.

Tables 7 and 8 display the results obtained with SC+ panels tested with two SAR-COVID-19 surrogates (HCoV-E229 and HCoV-OC43). As can be seen in these tables, the SC+ effectively deactivated both type of virus strains after 4-h of exposure. The experimental findings demonstrated that it is possible to inactivate coronavirus surrogates by means of the reported mechanism of degrading viral proteins by ROS species under the UV intensity and irradiation times applied in this investigation. Moreover, it is expected that SC+ surfaces will be able a further deactivate other microorganism as have been reported for model airborne viruses and bacteria like E. coli.

TABLE 7
ISO 18061-2014 results for human coronavirus 229E (HCoV-229E)

| | | | Infectivity | UV | UV | Percentage |
|---|---|---|---|---|---|---|
| | | Cell | Titer | intensity | radiation | reduction |
| Sample | Virus | line | TCID50/ml | (mW/cm²) | time (h) | * |
| 1 | HCoV- | MRC-5 | 10⁵ | 0.25 | 4 | 100% |
| 2 | 229E | | | | | 99% |
| 3 | | | | | | 100% |

* Percentage reduction calculated per original infectivity titer of virus inoculum.

TABLE 8
ISO 18061-2014 results for human coronavirus OC43 (HCoV-OC43)

| | | | Infectivity | UV | UV | Percentage |
|---|---|---|---|---|---|---|
| | | Cell | Titer | intensity | radiation | reduction |
| Sample | Virus | line | TCID50/ml | (mW/cm²) | time (h) | * |
| 1 | HCoVO- | HCT-8 | 10⁵ | 0.25 | 4 | 100% |
| 2 | C43 | | | | | 100% |
| 3 | | | | | | 100% |

* Percentage reduction calculated per original infectivity titer of virus inoculum.

### TAKTL SC+ Panel Moss Growth Evaluation

Another important factor in the performance of a building façade is maintenance during its service life. In this regard, buildings located in humid climates like the Pacific Northwest, present the significant problem of biofouling or the growth of mold, algae, and moss on the surface. Mold can be found everywhere and can grow on any material in the presence of moisture. Moss growth is typically more of a problem in outdoor environments except in case of a water leaks or very humid indoor environments such as atriums. The growth of the mold and moss occurs in the presence of porosity in the building materials that allow spores to lodge and grow. When the spores land on a building facade they start to reproduce all over the material and in some cases are very difficult to clean. Thus, there has been a need to develop façade panels that prevent biofouling on their surface. As in the case of the antimicrobial properties demonstrated with SC+ panels in previous sections, it was expected that those same ROS effects will help to decrease or inactivate the mold by preventing moss spores' survival. This section evaluates moss growth on the surface of SC+ panels under conditions controlled to encourage its growth.

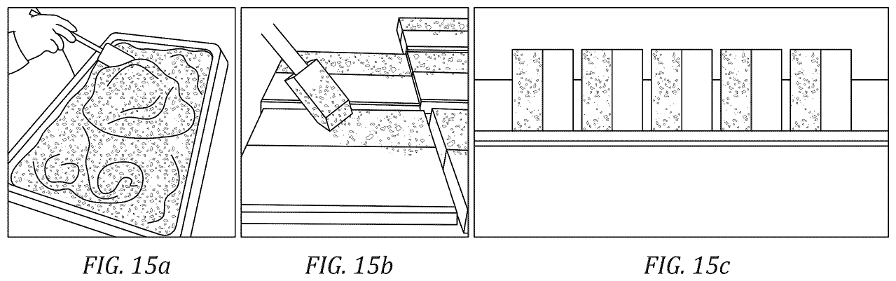

Several 6×4 in. TAKTL SC+ W10 (Mix-B) panels with Rough 1, cast and media-blast textures of diverse colors were used for the experiment (WH87, BO78, PL75, TI63 and TE52). A commercial mix of several moss spores was used. The common method used to grow moss in landscape architecture was employed to prepare the moss culture media. Equal amount of water and buttermilk were measured and blended in a pan-type container (FIG. 15a).

All ingredients were blended until a fine liquid mixture was achieved. Later, the mixture was painted in two layers on one half of each SC+ sample surface using a paintbrush (FIG. 15b). After air-drying, samples were stored horizontally on a support in a moisturized curing room kept at 80 F and 80% humidity, and with a fluorescent light emitting UV-A radiation (FIG. 15c). Once a day, the surface of the panels was sprayed with water to maintain the water-saturated surface. Samples were tested for a period of 15 months.

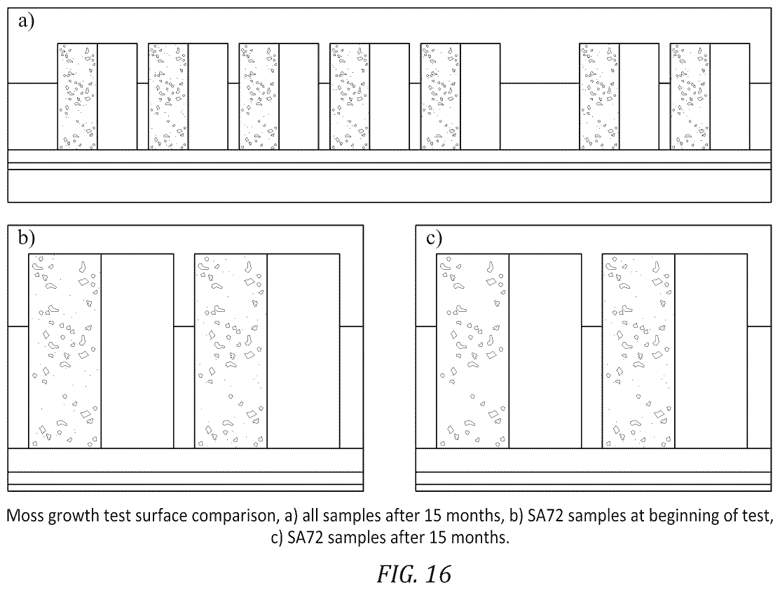

FIG. 16 shows the appearance of the panels after 15 months in the controlled heat and humidity room.

As can be seen in this figure, no moss growth is observed. On some samples, the buttermilk layer is not appreciable and a portion of the spores on the surface have been removed, as expected, since the ROS species inactivates any reproduction of spores, thus preventing moss growth. It is important to notice that, in general, standard UHPC surfaces are not an ideal environment for biofouling in any case, since UHPC is by definition a concrete with very low porosity and high alkalinity. Therefore, to complement this test series, a standard mold test such as ASTM G21 could be performed to verify the results. However, based on the moss and antiviral tests in this report and as demonstrated in other building materials doped with photocatalytic nanoparticles, it is expected that SC+ panels will be resistant to biofouling under light conditions that support photocatalytic activity.

### Color and Weatherabilty (ASTM G155 and ASTM D2244)

In previous sections, the photocatalytic activity of SC+ panels have been demonstrated, based upon different model pollutant and microorganism resistance. Other important aspects of façade panel performance are their aesthetics and the preservation of color over time. This test series is designed to determine the self-cleaning effect and the weatherability of architectural concrete through its resistance to the color fade of inorganic pigments and the formation of efflorescence.

To determine the resistance to efflorescence and color change caused by wet and dry cycles under the presence of light, two distinct evaluation methods were applied: 1) accelerated weathering test and 2) natural outdoor exposure.

### TAKTL SC+ Panel Accelerated Weather Testing (ASTM G155)

For the determination of the effects on color and efflorescence potential under simulated weathering conditions, a Q-SUN Xe-2 Xenon Arc light weathering machine was used. The ASTM G155 standard method was applied using the exposure cycle 1. On this cycle a daylight filter with a wavelength of 340 nm and a light irradiance of 0.35 W/m²·nm were applied to several rough 1 and media-blast 1.75×2 in. panel samples sealed with Microseal. Five colors (conventional and SC+ panels) were selected for the evaluation, WH87, BO78, PL75, SA72 and TI63.Over a total 500 hours of exposure, the panels were subjected to repeating 2-h cycles set at 102 min of light at 63° C. of black panel temperature followed by 18 min of light and water spray (air temperature not controlled). Color values before and after were measured by a Datacolor G45 Color photo spectrometer which provide color values using CIELAB color space (L, a, b), where L is the lightness of the color from Black (0) to White (100), a, is the tone from red (+50) to green (−50) and b is the tone from yellow (+50) to blue (−50). Color difference between the un-exposed and exposed samples were determined following ASTM D2244, which defines color difference ΔE as:

$$Δ E = {[ {( Δ L )}^{2} + {( Δ a )}^{2} + {( Δ b )}^{2} ]}^{1 / 2} \qquad Eq . 7$$

Where ΔL, Δa and Δb are the differences between the reference un-exposed color coordinates and the final values after 500-h of accelerated weathering exposure.

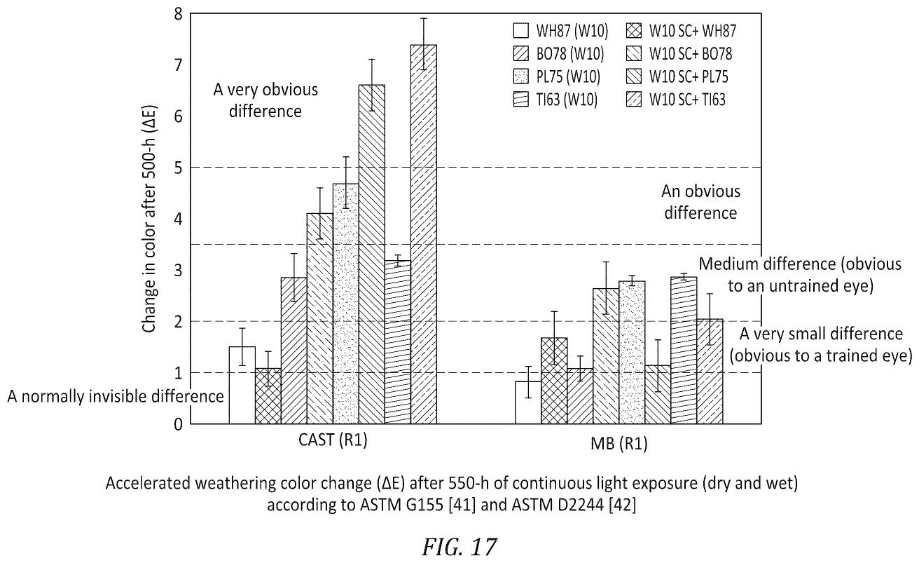

The results obtained for the accelerated weathering are displayed on FIGS. 17 to 19.

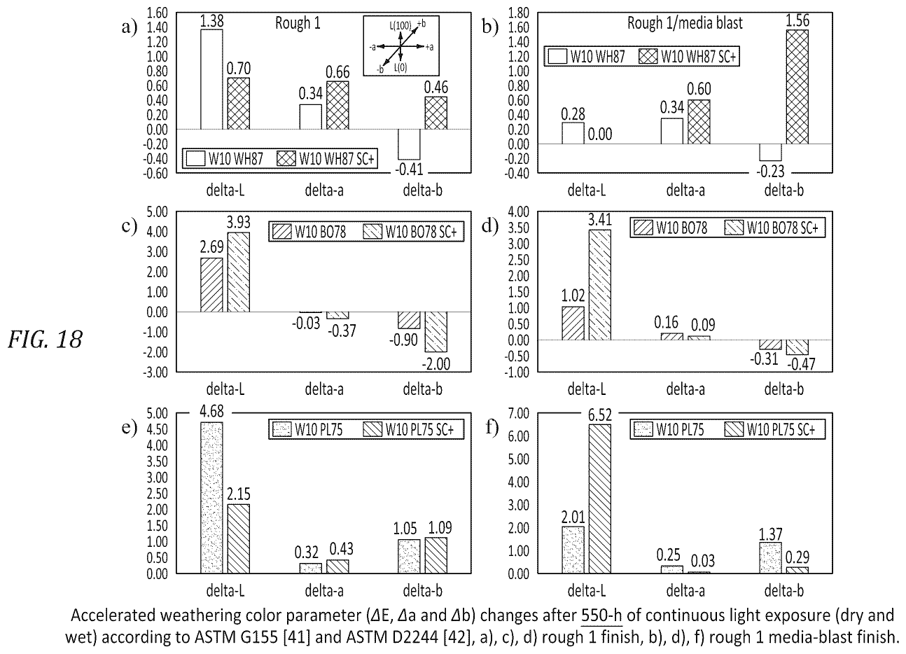

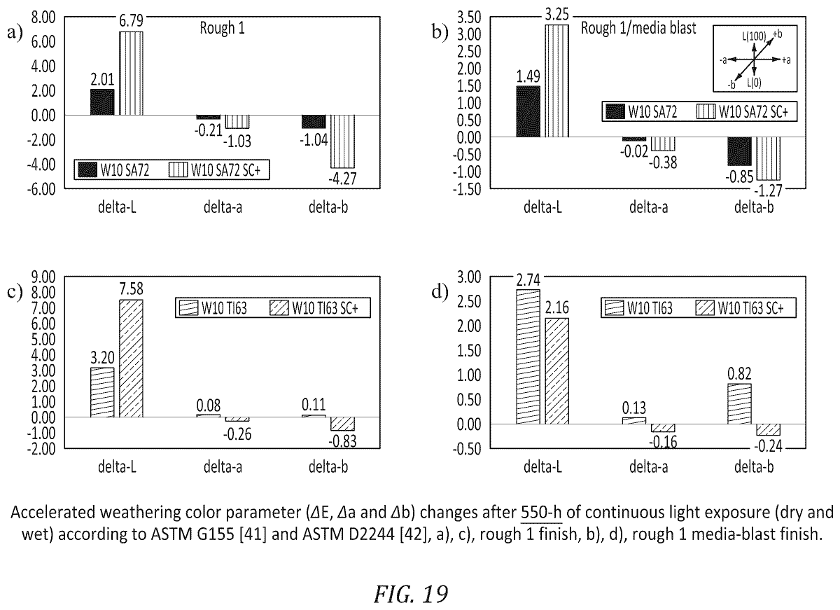

As can be seen in FIG. 17, the total change in color for a majority of SC+ panels were greater than the reference panel. This result is expected as this test involves light on during all the cycles and total exposure time. There is no period for the material to relax and further dissipate ROS during the dark period that occurs in normal outdoor conditions. As a result, the photocatalytic effect is acting at all times on the surface, producing electron-holes pairs that interact with the concrete pigments, thereby inducing an acceleration of the natural color fading that is further enhanced by dry and wet events. All samples, conventional and SC+, lighten to some degree (FIGS. 18 and 19), with the degree of color fade highest in darker colors and cast (vs. mediablast) surfaces which are rich on fine particles.

This result is expected, since it has been reported that iron base pigments (Fe₂O₃, Fe₃O₄ and FeOOH) present electronic interaction (limiting or enhancing the e⁻-h· pairs recombination) with photocatalytic pigments as they are also semiconductors in origin as Fe₂O₃ which present catalytic effects on visible light. These electronic interactions produced an accelerated oxidation of the pigments producing color fading and delaying the self-cleaning reaction. This is confirmed observing that the SC+ WH87 panels showed in some cases lower L value and higher b values characterized by degradation or organic pollutants (yellowing).

Similarly, yellow pigments (FeOOH) have been reported to leach more quickly to the surface as a result of its lighter density. It is believed that yellow pigments reacted with the reaction product from the environment, possibly producing calcium and iron nitrates or carbonates minerals that are easily washed out by the action of the water spray, which would explain the decreasing in b values for BO78 and SA72 color panels.

Despite the accelerated weathering results showing faster color fading under the accelerated test conditions, the results demonstrated one more time the photocatalytic properties of SC+ panels surface. However, based on these results it is expected that the self-cleaning effects would be higher on light color panels and slightly decreased on dark saturated or high yellow color that specifically contain high amounts of red iron oxide (Fe₂O₃) and yellow (FeOOH) pigments. Finally, it is important to note that no cracks, chalking or dusting of the panels were observed.

### TAKTL SC+ Panel Natural Outdoor Weathering

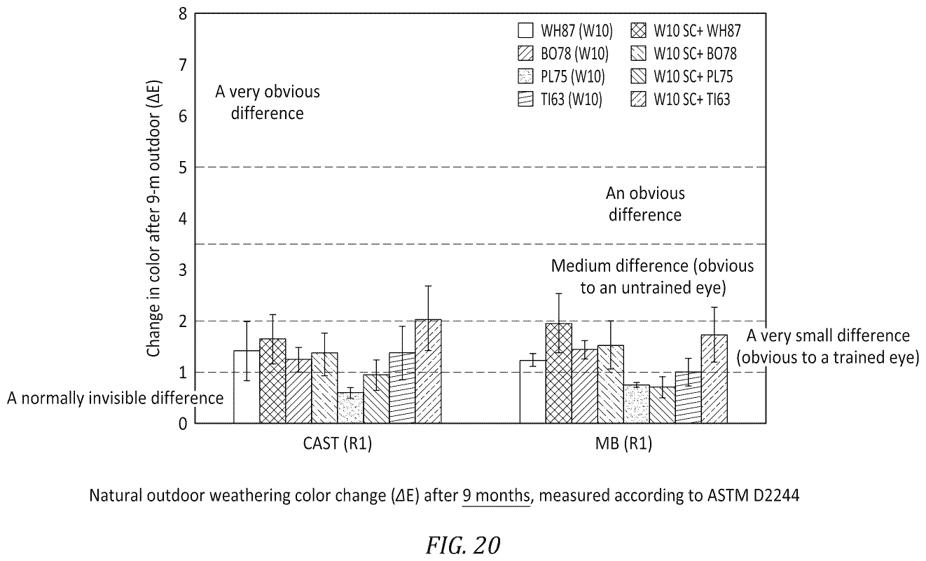

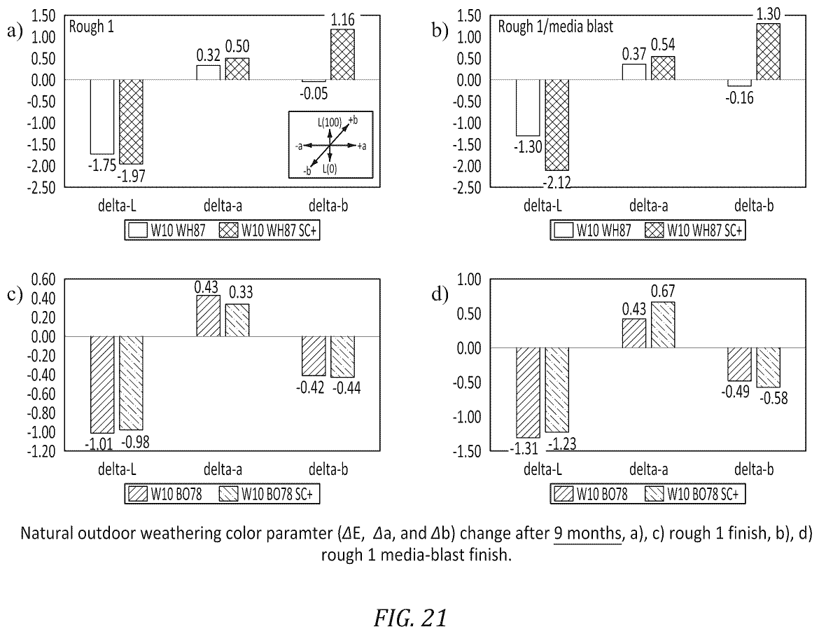

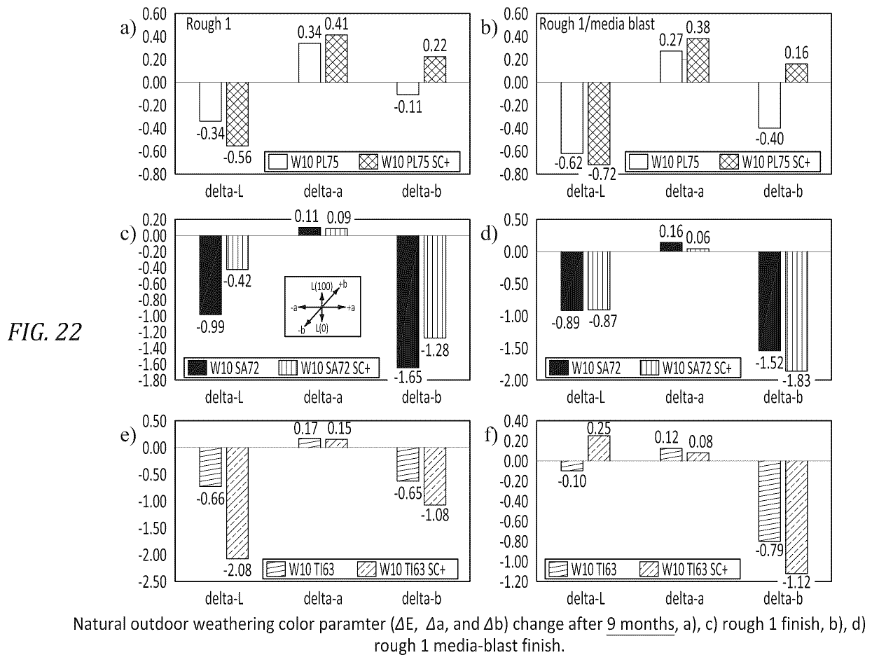

The same color samples tested in the last section (WH87, BO78, PL75, SA72 and T163) were used for outdoor natural exposure tests. Conventional and SC+ 2×14 in. rough 1 samples with half of the surface mediablast and both sides having a Microseal finish were installed on TAKTL's outdoor testing rack located in Turtle Creek, PA for a period of 9 months. Initial color and color after exposure were measured again using a Datacolor G45 color photo spectrometer, with the color change calculated based on the ASTM D2244 standard. FIG. 20 displays the total color change (ΔE) after 9 months of environmental exposure. As shown in this figure, the SC+ showed similar or marginally higher color change (most noticeable on TI63 panels) compared with conventional ones. Nevertheless, the color differences are almost imperceptible, with ΔE lower than 2points and inside of the experimental variation band of the test (±2δ). The primary differences can be seen on changes related to L, a and b color parameters shown in FIGS. 21 and 22.

Contrary to the results accelerated weather conditions in which a lightening of the panels was evident, under natural exposure conditions, a darkening or decrease on L values was actually observed for all panels, except for only the TI63 SC+ panel (gray) with mediablast finish that produced a ΔL of 0.25 points (lower than standard error). Slight darkening of the panels is expected as the hydration degree of the concrete panels surface increases with time, changing the color saturation and normally darkening the tone. Again, different color changes are also observed which were color and pigment depended. For WH87 panel tone (white), the SC+ showed the already explained effects of yellowing due to the degradation of pollutant on the surface (increase in a and b scale). The increase on a and b scale is also reflected on the slight decrease on L values.

This is direct evidence of the photocatalytic effects on the surface. Similar behavior was observed for SC+ panels with PL75 tones (very light gray). For panels with colors that use yellow pigment on their composition (BO78, SA72 and TI63) a decrease in yellowness is evident and, in some cases, there is no appreciable color difference between conventional panels and SC+ (case BO78 and SA72). The reasons for this result have been explained previously herein, namely, the leaching and mineralization of iron hydroxide (FeOOH) particles from the surface due to their lighter density compared with other types of pigments. As explained on the ISO 10678 tests performed, the presence of iron-based pigments appears to delay or interfere to some degree with the self-cleaning effects, as compared to panels made with WH87 white pigments (FIG. 7). Based on this observation, it is expected that panels with lighter colors will perform more efficiently than panels composed of darker colors which contain higher amounts of pigment.

Based on the accelerated and natural outdoor weathering testing, it is expected that the color of SC+ panels would be better preserved over a longer period than non-SC+ panels due to their self-cleaning and pollutant degradation properties. The precise long-term color variation is still unknown, but it is not expected that the color change surpasses the AAMA 2605-13 [43] specification of a maximum ΔE of 5 points after 10 years of natural exposure since the observed color changes appear to stabilize after 9 months of outdoor conditions. No cracks, chalking or visual degradation have been observed during the period of the test.

1. A composite ultra-high-performance concrete mixture, comprising:
cement in an amount between 500 and 680 kg/m³; a photocatalyst in amount between 10 and 30 kg/m³; a recycled glass white nano-silica in amount 40 and 60 kg/m³; calcinated kaolin in amount 70 to 120 kg/m³; a filler in amount between 200 and 300 kg/m³; a fine sand in amount between 600 to 970 kg/m³; and a coarse sand in amount between 400 to 600 kg/m³.

2. The concrete of claim 1, wherein the cement comprises cement particles having an average particle size of less than 90 μm.

3. The concrete of claim 1, wherein the cement has a specific surface area less than 2.0 m²/g.

4. The concrete of claim 1, wherein the cement has a Blaine fineness of less than 510 m²/kg.

5. The concrete of claim 1, wherein the photocatalyst is in powder form with at least 988 wt Anatase base TiO₂ with B.E.T. surface area between 50 and 400 m²/g.

6. The concrete of claim 1, wherein the recycled glass white nano-silica has a minimum of 99.0% amorphous SiO₂ content with B.E.T. surface area between 30 and 60 m²/g and primary particle size between 30 and 300 nanometers.

7. The concrete of claim 1, wherein the calcinated kaolin has particle size between 1 to 7 microns and B.E.T. surface area between 2 to 10 m²/g.

8. The concrete of claim 1, further comprising Nepheline-Syenite filler, and fine and coarse sand.

9. The concrete of claim 8, wherein the Nepheline-Syenite filler is present in an amount between 200 and 300 kg/m³ and has a particle size less than 75 μm.

10. The concrete of claim 1, further comprising Nepheline-Syenite fine sand.

11. The concrete of claim 10, wherein the Nepheline-Syenite fine sand is present in an amount between 600 and 970 kg/m³ and has a particle size less than 600 μm.

12. The concrete of claim 1, further comprising Nepheline-Syenite coarse sand.

13. The concrete of claim 12, wherein the Nepheline-Syenite coarse sand is present in an amount between 400 and 500 kg/m³ and has a particle size less than 1500 μm.

14. The concrete of claim 1, further comprising fibers selected from the group consisting of steel fibers, natural fibers, synthetic fibers and mixtures thereof.

15. The concrete of claim 1, further comprising a total water content of between 150 and 275 kg/m³.

16. The concrete of claim 1, wherein the concrete exhibits a compressive strength between 90 MPa and 120 MPa in a normal curing regime.

17. The concrete of claim 1, wherein the concrete exhibits a three bending point flexural strength of between 10 and 35 MPa.

18. The concrete of claim 1, wherein the concrete exhibits a slump flow of between 250 and 350 mm.

19. A method for manufacturing an enhanced photocatalytic concrete panel, comprising the steps of:
sprinkling clear glass and marble aggregates into a urethane resin mold; spreading a first concrete layer over the aggregates; setting an AR-glass mesh on the first concrete layer; spreading a second concrete layer on top of the AR-glass mesh; setting a second AR-glass mesh on the second concrete layer; and spreading a third concrete layer over the second AR-glass mesh to form a composite panel.

20. The method of claim 19, wherein the clear glass is recycled clear window or municipal waste flint class with aggregate size between 1 and 7 mm.
